(19) United States
(12) Patent Application Publication
(10) Pub. No.: US 2009/0186835 A1
(43) Pub. Date: Jul. 23, 2009

(54) TREATMENT AND PROPHYLAXIS OF CANCER

(76) Inventors: Richard A. Gross, New York, NY (US); Martin H. Bluth, Detroit, MI (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/353,571

(22) Filed: Jan. 14, 2009

Related U.S. Application Data
(60) Provisional application No. 61/021,109, filed on Jan. 15, 2008.

(51) Int. Cl. A61K 31/7028; A61P 35/00

(52) U.S. Cl. 514/25

(57) ABSTRACT

A method and composition for the prophylaxis or treatment of humans or animals for cancer using a mixture of sophorolipids.

### STATEMENT OF RELATED APPLICATIONS

This application claims the benefit of U.S. Provisional Patent Application No. 61/021,109, filed on 15 Jan. 2008, which is incorporated herein in its entirety by this reference.

### BACKGROUND OF THE INVENTION

1. Technical Field

The present invention relates generally to the field of sophorolipids and more specifically to the field of using sophorolipids as anticancer and cancer treatment agents and for the prophylaxis of cancer.

2. Prior Art

Cancer is a group of diseases in which cells are aggressive (grow and divide without respect to normal limits), invasive (invade and destroy adjacent tissues), and sometimes metastatic (spread to other locations in the body). Cancer may affect people at all ages, but the risk for the more common varieties tends to increase with age.

Most cancers can be treated or can be cured, depending on the specific type, location, and stage. After diagnosis, cancer usually is treated with a combination of surgery, chemotherapy and radiotherapy. As research develops, treatments are becoming more specific for different varieties of cancer. There has been significant progress in the development of targeted therapy drugs that act specifically on detectable molecular abnormalities in certain tumors, and which minimize damage to normal cells. The prognosis of cancer patients is most influenced by the type of cancer, as well as the stage, or extent of the disease.

In one example, pancreatic cancer continues to be one of the leading causes of cancer-related deaths in the US. Even with surgical treatment and numerous recent therapeutic interventions, such as gemcitabine and topo-isomerases, there is still no treatment that significantly impacts the death toll. Even though specialty surgery with adjuvant chemotherapy results in significant advantages in terms of 5-year survival rates, adjuvant systemic 5-fluorouracil with folinic acid improves 5-year survival by only 29% and adjuvant gemcitabine improves disease-free survival only to 13.4 months. Therefore, it is ever so important to find better methods of treatment of this disease.

Glycolipids and their derivatives are of great interest in malignancy and other disorders because of their varied biological activities and potential for therapeutic uses. These anticancer effects have been reported for lung, cervical, breast, and brain cancers and have been shown to use mechanisms including regulation of angiogenesis and apoptosis, among others. Sophorolipids are unique members of the glycolipid family, in that they are easily chemoenzymatically modifiable, thereby providing individual treatment possibilities in different diseases. In this regard, sophorolipids or select derivatives possess antimicrobial, antiviral, and anti-inflammatory properties. Sophorolipids have been shown to possess anticancer effects against hepatocellular carcinoma.

Accordingly, there is a need for an improved method for inhibiting and killing cancer cells. There is also a need for a treatment of cancer in mammalian subjects. It is to these needs, among others, that this invention is directed.

### BRIEF SUMMARY OF THE INVENTION

A method for the prophylaxis or treatment of cancer in humans and other host animals comprising the administration of sophorolipids or sophorolipid analogs in a pharmaceutically acceptable carrier. The method demonstrates sophorolipid-mediated cytotoxic responses to cancer cell lines. These anticancer responses were dose- and derivative-dependent and likely kill cancer cells by necrosis. Furthermore, these agents are specific to cancer cells in that they did not affect normal human cells. Therefore, sophorolipids represent a unique and novel class of drugs, which may be an important promising therapy against cancer.

Human pancreatic carcinoma (HPAC) cells (1×10⁴/ml) were treated with increasing concentrations (0.5, 1.0, 1.5, and 2.0 mg/ml) of a sophorolipid natural mixture and six select sophorolipid derivatives (ethyl ester, ethyl ester monacetate, ethyl ester diacetate, methyl ester, acidic sophorolipid, and lactonic sophorolipid diacetate) and were assessed for cell necrosis (cytoxicity—LDH release) and apoptosis (annexin). Controls consisted of cells treated with media or vehicle alone and sophorolipids treatment of peripheral blood mononuclear cells (PBMC).

Analysis of dose-dependent responses demonstrated that sophorolipid derivatives differed in their ability to kill cancer cells. Although the cytotoxic responses of many of the derivatives tested were positively dose dependent, some derivatives, i.e., LSD and AS, showed responses which were inversely proportional to dose. As the lactonic acid form (LSD) is a closed ring structure, this difference in conformation may play a role in its decreased cytotoxic activity at higher doses as it may become supersaturated. Alternatively, increased dosing may down-regulate the anticancer mechanism responsible for the antitumor effect. The ME derivative, which was the most potent among all of the derivatives, showed roughly the same cytotoxicity at all doses. It is likely that this derivative utilizes a mechanism distinct from the other derivatives, which facilitates the same degree of cytotoxicity upon activation.

### BRIEF DESCRIPTION OF THE FIGURES

FIG. 1A shows the basic structure of sophorolipids suitable for use in the present invention.

FIG. 1B shows the basic structure of sophorolipid analogs suitable for use in the present invention.

FIG. 2 shows the cytoxicity of sophorolipid and derivatives.

FIG. 3 shows the cytotoxicity with lactonic sophorolipid diacetate derivative.

FIG. 4 shows the cytotoxicity with sophorolipid methyl ester derivative.

FIG. 5 shows the cytotoxicity with sophorolipid ethyl ester diacetate derivative.

### DETAILED DESCRIPTION OF PREFERRED EMBODIMENTS

Embodiments of this invention are based on the discovery that the administration of sophorolipids, sophorolipid analogs, and mixtures thereof can provide aid in the prophylaxis and treatment of cancer. Embodiments of this invention are mixtures of sophorolipids and methods of using such mixtures for the prophylaxis or treatment of cancer or uncontrolled cell growth in animals and humans.

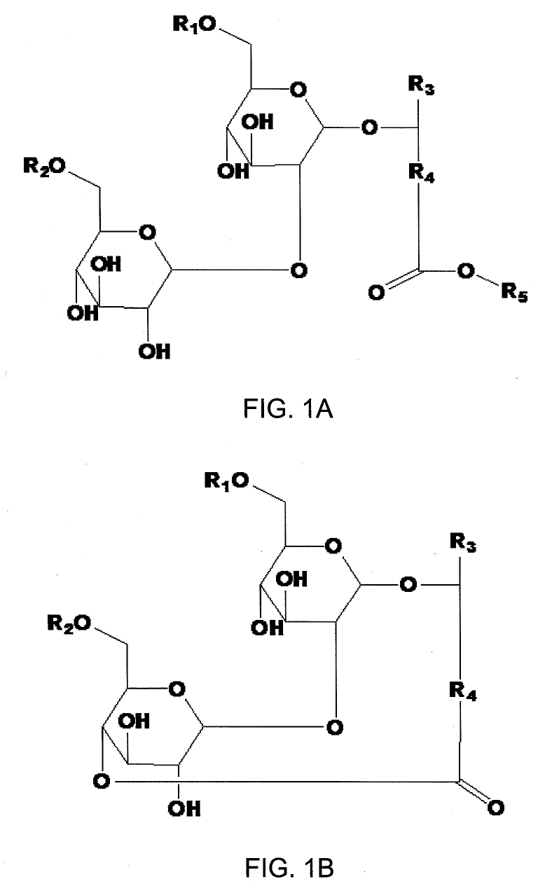

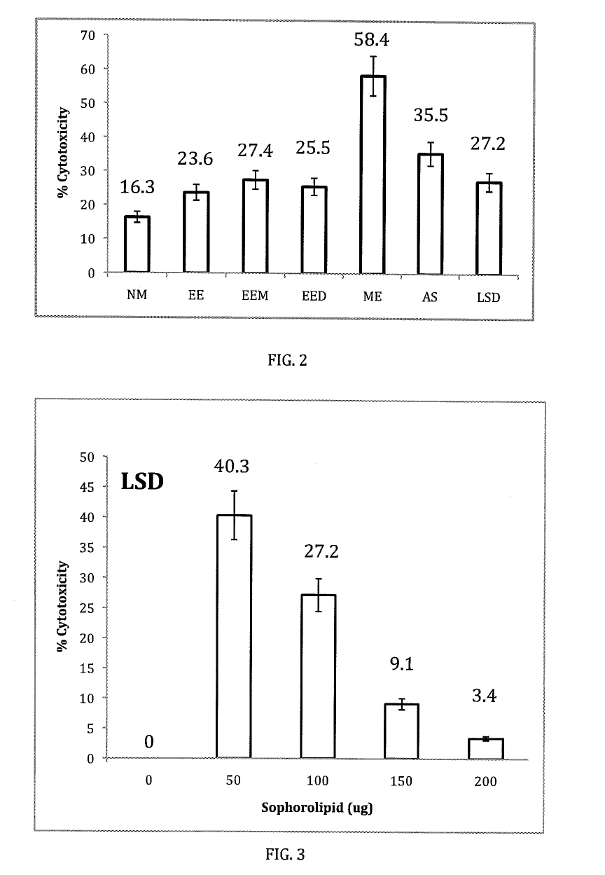

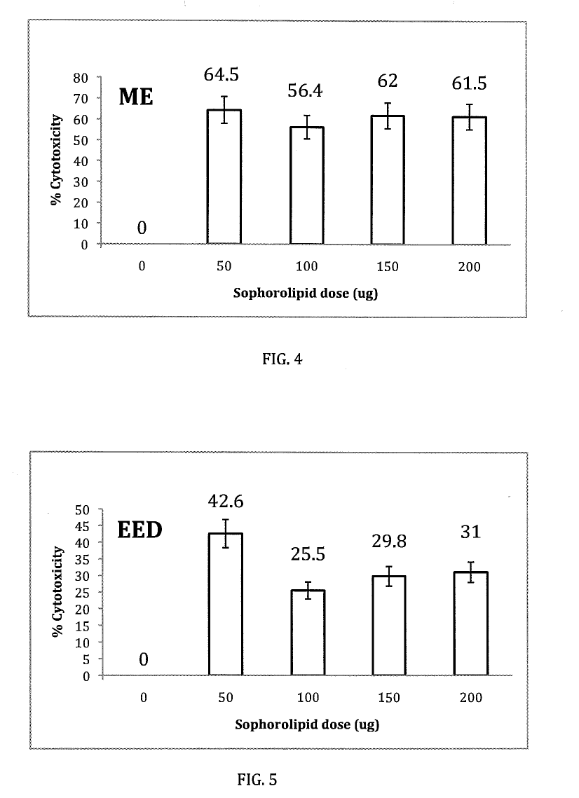

FIGS. 1A and 1B show the basic structures of sophorolipids and sophorolipid analogs suitable for use in the present invention. FIGS. 2-5 show the cytotoxicity of various sophorolipids and derivatives to human pancreatic carcinoma cells (HPAC). Specifically, FIG. 2 shows the cytoxicity of sophorolipid and derivatives, FIG. 3 shows the cytotoxicity with lactonic sophorolipid diacetate derivative, FIG. 4 shows the cytotoxicity with sophorolipid methyl ester derivative, and FIG. 5 shows the cytotoxicity with sophorolipid ethyl ester diacetate derivative.

Suitable sophorolipids and sophorolipid analogs include those having the formulas shown in FIGS. 1A and 1B. In one embodiment, R₁ and/or R₂ represents hydrogen or an acetyl group; R₃ can be a hydrogen or a methyl group; R₄ is an alkyl chain that normally has 15 carbons but can have between 9 and 19 carbons and normally has one site that is unsaturated (C═C bond); and R₅ normally consists of a H or an n-alkyl group with a chain length from 1 to 12 carbons (methyl to dodecyl). In another embodiment, R₁ and/or R₂ can have longer acyl groups that include C3-C18 units that can be branched, unsaturated, have epoxy moities or hydroxyl groups. In another embodiment, R₄ can have multiple sites of unsaturation, other elements within chains such as oxygen (e.g. CH₂—O—CH₂), sulfur (e.g. CH₂—S—CH₂), fluorine (CF₂) segments, and/or phenyl moities. In another embodiment, R₅ can be a group that in itself provides bioactivity. Examples include R₅ groups that are steroids, anti-inflammatory compounds, and/or anti-cancer molecules such as taxol and oligopeptides with various biological activities. Exemplary compounds are shown in Appendix A.

One embodiment includes a method comprising the administration of a sophorolipid mixture in a therapeutically effective amount to humans or animals in which the sophorolipid mixture contains compounds having the formula shown in FIG. 1A or 1B. It is understood that the sophorolipids also can be deacetylated and esterified at the carboxylic group or groups so that the sophorolipid can be used as a therapeutically active substance in a therapeutic treatment method suitable for the human or animal body. It is contemplated that different structural sophorolipid derivatives and mixtures thereof can have different levels of biological activity. Exemplary esterifications and deacetylations are shown in Appendix B.

The amount of the sophorolipid composition administered, namely the therapeutically effective amount, is an amount sufficient to induce or produce an anti-cancer effect in the patient. A “therapeutically effective amount” in the context of treating tumors and cancer, refers to an amount capable of inhibiting tumor growth; reducing tumor size; inhibiting tumor cell infiltration into peripheral organs; and/or inhibiting metastasis. Further this term can include an amount capable of enhancing the anti-tumor immune response and relieving one or more symptoms associated with the cancer or tumor.

The sophorolipids and/or sophorolipid analogs can be administered to a patient at a recommended dose, which can be the highest dose able to be safely administered to a patient. Those having ordinary skill in the art can ascertain the most effective dose and times for administering the agents, considering route of delivery, metabolism of the compound, and other pharmacokinetic parameters such as volume of distribution, clearance, age of the subject, and other factors, without undue experimentation. In one example, a therapeutically effective amount can range from about 0.1 to 1000 mg/kg body weight.

In one embodiment, the sophorolipids can be synthesized by fermentation of Candida bombicola. After fermentation, the sophorolipids can be extracted using, e.g., ethyl acetate. The extracts can be pooled and the solvent then can be removed. Residual fatty acids can be removed by washing the product, e.g., with hexane. This product can be referred to as a natural sophorolipid. The sophorolipid then can be dried in a vacuum desiccators. The lactonic sophorolipids can be separated from the acid sophorolipids. Such a separation may be necessary in the event that one group of Sophorolipids or one derivative may have an increased benefit as an anti-cancer agent than the natural mixture or another group or another one of the derivatives.

One embodiment of this invention includes the use of a natural mixture of lactonic and esterified sophorolipids as anticancer agents. The administration of lactonic sophorolipids, esterified sophorolipids, natural mixtures of sophorolipids, select derivatives of sophorolipids either individually or in combinations, and various other mixtures and combinations of sophorolipids and derivatives may have increased anti-cancer effects and that these effects may be dependent on the particular cancer or patient to be treated.

It is understood that the sophorolipids and/or sophorolipid analogs can be combined with other anticancer agents to prepare improved anticancer agents and medicines. Administration “in combination with” one or more further therapeutic agents includes simultaneous (concurrent) and consecutive administration in any order.

Those of ordinary skill in the art know techniques and general formulations suitable for administering the sophorolipid compositions. For example, such known techniques and formulations may be dependent on the method of administration, such as, for example, intravenous, intraperitoneal, subcutaneous, and other known routes of administration, as disclosed herein.

Those of ordinary skill in the art can choose other delivery systems and formulate the novel sophorolipid into the delivery system chosen without undue experimentation. The invention also relates to pharmaceutical compositions that are characterized in that they contain a pharmaceutically inert excipient and as an active ingredient contain at least one sophorolipid. The sophorolipid mixtures of the present invention can be mixed with a pharmaceutically acceptable carrier such as, for example purposes only, physiologically compatible buffers such as solution, physiological saline, a mixture consisting of saline and glucose, heparinized sodium-citrate-citric acid-dextrose solution, and other such acceptable carriers.

The active compounds also can be administered intraperitoneally. Solutions of the active compounds as freebase or pharmacologically acceptable salts can be prepared in water suitably mixed with a surfactant, such as hydroxypropylcellulose. Dispersions also can be prepared in glycerol, liquid polyethylene glycols, and mixtures thereof, and in oils. Under ordinary conditions of storage and use, these preparations can contain a preservative to prevent the growth of microorganisms.

Sterile injectable solutions can be prepared by incorporating the active compounds in the required amount in the appropriate solvent with various other ingredients, as needed, followed by filtered sterilization. Generally, dispersions can be prepared by incorporating the various sterilized active ingredients into a sterile vehicle that contains the basic dispersion medium and any other required ingredients from those enumerated above. In the case of sterile powders for the preparation of sterile injectable solutions, methods of preparation include vacuum drying and freeze-drying techniques that yield a powder of the active ingredient plus any additional desired ingredient from a previously sterile-filtered solution thereof.

It is understood that administration of sophorolipids and sophorolipid analogs can be effective against a wide variety of cancers. In this embodiment, the compound can be used to treat cancers of the prostate, pancreas, ovary, breast, myeloma, squamous cell carcinoma, and others. The sophorolipids have activity against cancer cells and exhibits low toxicity toward healthy cells. Previous studies have indicated that sophorolipids can be administered to subjects without undue toxicity.

### Example 1

### Summary

The sophorolipid mixtures of the present invention have anti cancer properties, which were confirmed by experiment and observations. Human pancreatic carcinoma (HPAC) cells (1×10⁴/ml) were treated with increasing concentrations (0.5, 1.0, 1.5, and 2.0 mg/ml) of a sophorolipid natural mixture and six select sophorolipid derivatives (ethyl ester, ethyl ester monacetate, ethyl ester diacetate, methyl ester, acidic sophorolipid, lactonic sophorolipid diacetate) for 24 hours and were assessed for cell necrosis (cytoxicity—LDH release) and apoptosis (annexin). Controls consisted of cells treated with media or vehicle alone and sophorolipids treatment of peripheral blood mononuclear cells (PBMC). AS mediated toxicity was inversely proportional with dose (LSD—40.3% at 0.5 mg/ml, 3.4% at 2.0 mg/ml, AS 49% at 0.5 mg/ml, 0% at 2.0 mg/ml). Dose dependant apoptosis could be observed with the AS derivative. Sophorolipid treatment did not effect PBMC at all doses tested.

### Materials and Methods.

Sophorolipids were synthesized by fermentation in Candida bombicola. The fermentation media contained glucose (100 g), yeast extract (10 g), ureas (1 g), and oleic acid (40 g) per 1000 mL water. After 7 days of fermentation, sophorolipids were extracted three times with ethyl acetate. The extracts were pooled and the solvent was removed. The obtained product was washed with hexane to remove residual fatty acids. Liquid chromatography/mass spectrometry and nuclear magnetic resonance analyses were carried out to verify the purity of the compounds. No residual fatty acids or media components were found in the sophorolipids. Sophorolipid derivatives, including ethyl ester (EE), ethyl ester monoacetate, ethyl ester diacetate (EED), methyl ester (ME), acidic sophorolipid (AS), and lactonic sophorolipid diacetate (LSD) (FIG. 1), were also studied to determine which derivative may work more effectively. Briefly, lactonic sophorolipids were purified from natural mixture using flash chromatography. Alcholysis reaction of natural mixture was carried out using sodium ethoxide to synthesize ethyl ester sophorolipids.

A pancreatic cancer cell line was selected as the representative cancer cell for illustrating the efficacy of this invention. Other cancer cell lines, such as but not limited to ovarian, prostate, brain, lung, cervical, breast, and multiple myeloma also could have been selected as the illustrative cell line. The pancreatic cancer cell line, human pancreatic carcinoma cells (HPAC), was obtained from American Tissue and Cell Culture and cultured in complete media according to American Tissue and Cell Culture guidelines in T-75-cm² flasks in a 37° C. incubator with 5% CO₂. HPAC cells were grown in Dulbecco's modified Eagle's medium with Ham F-12, 5% fetal bovine serum, 5% penicillin/streptomycin, 0.002 mg/mL insulin, 0.005 mg/mL transferrin, 40 ng/mL hydrocortisone, and 10 ng/mL epidermal growth factor.

1×10⁴ HPAC cells were aliquoted into each well of a 96-well plate and served as target cells. They were plated in phenol red dye-free RPMI 1640 (HyClone, Logan, Utah) complete medium (1 mM L-glutamine, 100 U/mL penicillin, 100 μg/mL streptomycin, 5% heat-inactivated fetal bovine serum; Gibco-BRL, Grand Island, N.Y.) and incubated with either natural mixture or sophorolipid derivative in increasing concentrations (0, 50, 100, 150, 200 mg; 100 μL final volume) at 37° C. with 5% CO₂ overnight.

Assays were carried out using a CytoTox 96 Non-Radioactive Cytotoxicity Assay (Promega, Woodshole, Mass.), in accordance with manufacturer's directions. This assay measures lactate dehydrogenase (LDH) release by target cells, after conversion of a tetrazolium salt into a formazan red product. Cytotoxicity as measured by LDH release is considered an effective measure of determining cellular necrosis. Sophorolipid mixtures or derivatives (0, 50, 100, 150, 200 mg) were added to 96-well tissue culture plates (Fisher Scientific, Springfield, N.J.) containing HPAC cells (100 μL final volume) and incubated at 37° C., 5% CO₂, for 20 h. To establish maximum lysis, lysing solution (9% v/v Triton-X 100; 10 μL) was added to representative culture wells containing HPAC cells. After incubation, supernatant (50 μL) was transferred to a fresh 96-well plate. Chromogenic substrate solution (50 μL) was added to each well while the plates were incubated in the dark at RT for 30 min, after which stop solution (1M NaAc; 50 μL) was added to each well to stop the reaction.

To determine the effect of sophorolipids on nonmalignant cells, sophorolipids were cultured in the presence of healthy peripheral blood mononuclear cells (PBMC). Cytotoxicity as measured by LDH release was determined by spectrophotometry at 450 nm (Bio-Tek Instruments Inc., Winooski, Vt.). When cells were cultured without sophorolipid, cell viability, as judged by Trypan blue dye exclusion, and cell recovery were >99%. Data shown represent the mean of at least three experiments±SE and are expressed as percentage cytotoxicity. Background controls were corrected for and consisted of target cell spontaneous (HPACs in media) and target cell maximum (HPACs in media with 10 μL lysis solution). Target cell maximum was corrected for by a volume control (media with 10 μL lysis solution). Each experiment was corrected for by a sophorolipid control (sophorolipid in media). These controls were incubated on the same 96-well plate as experimental assays. Percent (%) cytotoxicity was calculated according to the general equation below:

$$\% Cytotoxicity = \frac{Experimental - Target cell spontaneous }{Target cell maximum - Target cell spontaneous} \times 100$$

Values for results were expressed as means±SE obtained from multiple determinations in three or more separate experiments. P values computed were two-tailed, and P<0.05 was considered statistically significant (Student's t-test, analysis of variance with Tukey post-hoc correction; SPSS version 10.0, Chicago, Ill.).

### Results

When sophorolipid mixtures containing a combination of eight isoforms were cultured with HPAC cells, cytotoxicity was observed (FIG. 2). In addition, various degrees of cytotoxicity were observed with each derivative when compared with natural mixture: SL mixture=16.3%; EE=23.6%; EEM=27.4%; EED=25.5%; and LSD=26.2% (FIG. 2). Furthermore, cytotoxicity differed with sophorolipid dosing. LSD mediated decreasing cytotoxicity with increasing doses of sophorolipid (40.3% at 05. mg/mL, 3.4% at 2.0 mg/mL; FIG. 3). Similar responses were observed with AS (49% at 0.5 mg/mL, 0% at 2.0 mg/mL). ME derivative-mediated cytotoxicity was consistently elevated at all doses tested (63±5%; FIG. 4). EED derivative-mediated cytotoxicity was greatest at the lowest dose tested (50 μg) but maintained cytotoxic activity at all doses (36±6%; FIG. 5). No cytotoxicity was observed when similar doses of sophorolipid were cultured in the presence of PBMC.

FIG. 2 shows the cytotoxicity of sophorolipid and derivatives. HPAC cells were cultured with 100 μg of sophorolipid natural mixture or select derivatives. Cytotoxicity was determined as described in Materials and Methods above. Data are presented as mean of three experiments and are reported as percent cytotoxicity±SE and significance determined by Student's t-test; *P<0.05 compared with natural mixture (SL mix). SL mix=natural mixture; SL EE=ethyl ester; SL EEM=ethyl ester monoacetate; SL EED=ethyl ester diacetate; SL ME=methyl ester; AS=acidic sophorolipid; LSD=lactonic sophorolipid diacetate.

FIG. 3 shows the cytotoxicity with LSD derivative. HPAC cells were cultured with LSD derivative in increasing concentrations (50-200 μg). Cytotoxicity was determined as described in Materials and Methods above. *P<0.05 compared with no treatment; # compared with 50 μg treatment dose.

FIG. 4 shows the cytotoxicity with sophorolipid ME derivative. HPAC cells were cultured with ME derivative in increasing concentrations. Cytotoxicity was determined as described in Materials and Methods above. *P<0.05 compared with no treatment.

FIG. 5 shows the cytotoxicity with sophorolipid EED derivative. HPAC cells were cultured with EED derivative in increasing concentrations. Cytotoxicity was determined as described in Materials and Methods above. *P<0.05 compared with no treatment.

### Discussion.

Although glycolipids, including sophorolipids, have been gaining more interest in various disease states, to date, there have not been any studies on the effects of sophorolipids in pancreatic cancer. In studies demonstrating that sophorolipids had an effect against hepatocellular carcinoma, sophorolipids were found to induce apoptosis when cultured with human liver cancer cells (H7402). Sophorolipids are a unique type of glycolipid in that the disaccharide sophorose ring is glycosidically linked to the penultimate carbon of a long chain fatty acid (C₁₆ to C₁₉). Sophorolipid production occurs as a natural mixture, comprising many derivatives. The majority of these derivatives include either a 1′,4″ macrocyclic lactone ring (lactonic SLs) or a free carboxylic acid end (nonlactonic SLs). This overall construct provides the ability for sophorolipids to be chemoenzymatically modified at the carboxylic acid groups of each sugar moiety (R groups in FIG. 1). Furthermore, they can be produced in a number of different species including C. bombicola, Yarrowia lipolytica, Candida apicola, and Candida bogoriensis through fermentation of many different substrates, allowing for greater yield of material for modification and potential therapeutic application.

In this invention, natural mixture of sophorolipids and several of its derivatives demonstrate cytotoxicity in human pancreatic cancer cell lines by LDH release. This effect appears specific to malignant cells since no cytotoxicity was observed against normal human cells (PBMC). The ability to selectively affect malignant cells without harming normal cells would be advantageous in minimizing the side effects commonly associated with current therapeutic regimens. Cytotoxicity as determined by LDH release often involves necrosis as a mechanism of action.

Sophorolipids exist in a natural mixture, and, although there have been reports of their effects against malignancy, this is the first study to examine specific derivatives of sophorolipids against any cancer cell line. It has previously been demonstrated that certain sophorolipid derivatives did in fact differ in activity. In those studies, the EE and ME derivatives were able to decrease sepsis-related mortality and inflammatory cytokine production when compared with the natural mixture and other derivatives. Furthermore, no adverse effects were observed in vivo, suggesting that these agents are likely safe as a therapeutic modality. In the present invention, sophorolipids and their derivatives were able to kill cancer cells. However, ME demonstrated the most robust antitumor effect, which was maintained at all doses.

Analysis of dose-dependent responses demonstrated that sophorolipid derivatives differed in their ability to kill cancer cells. Although the cytotoxic responses of many of the derivatives tested were positively dose dependent, some derivatives, i.e., LSD and AS, showed responses which were inversely proportional to dose. It could be that, since the lactonic acid form (LSD) is a closed ring structure, this difference in conformation may play a role in its decreased cytotoxic activity at higher doses as it may become supersaturated. Alternatively, increased dosing may down-regulate the anticancer mechanism responsible for the antitumor effect. The ME derivative, which was the most potent among all of the derivatives, showed roughly the same cytotoxicity at all doses. It is likely that this derivative utilizes a mechanism distinct from the other derivatives, which facilitates the same degree of cytotoxicity upon activation.

### Example 2

Data demonstrates that sophorolipids are non-toxic in animal models at various doses. These results are shown in Appendix C.

Various cell lines including hey (ovarian), T47D (breast), LNCAP & PC-3 (both prostate), U266 (multiple myeloma) were tested. In order to ascertain the toxicity of sophorolipids, cells were ground in a NL media (100 mL/well at 20000 cells/mL) and were allowed to adhere for about 6-18 hours. Test samples were treated with a natural sophorolipid or sophorolipid derivative in an amount of about 0.1-40 mM (100 mL/well) and a control sample was established. The test sample and the control sample were monitored after 48 to 96 hours by SRB staining (protein) and with an ELISA reader at 570 nm. Before the drug media of adherent cells was replaced with serum free media, an additional 24 hours of incubation and an additional amount of drug was added at 0.1-10 mM. As shown in Appendix C, most cancer cell lines demonstrated increased cell death and/or apoptosis with increasing sophorolipid and/or derivative administration in a dose dependent fashion. This provides proof of concept for the use of sophorolipids and/or select derivatives administered either individually or in combination for use as anti-cancer agents.

The above detailed description of the embodiments, and the examples, are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention.

What is claimed is:

1. A method for prophylaxis or treatment of cancer in a biological environment comprising administering a therapeutically appropriate amount of a sophorolipid derivative or specific mixture of natural and/or modified sophorolipids to a human or animal.

2. The method as claimed in claim 1, wherein the biological environment is a mammal.

3. The method as claimed in claim 1, wherein the biological environment is a human.

4. The method as claimed in claim 1, wherein the sophorolipid derivative is selected from the group consisting of ethyl ester, ethyl ester monoacetate, ethyl ester diacetate, methyl ester, acidic sophorolipid, and lactonic sophorolipid diacetate.

5. The method as claimed in claim 1, wherein the mixture is administered by intravenous, intraperitoneal, subcutaneous, and other known routes of administration.

6. The method as claimed in claim 1, wherein the cancer is pancreatic cancer.

7. The method as claimed in claim 1, wherein the cancer is ovarian cancer.

8. The method as claimed in claim 1, wherein the cancer is breast cancer.

9. The method as claimed in claim 1, wherein the cancer is prostate cancer.

10. The method as claimed in claim 1, wherein the cancer is multiple myeloma cancer.
